(19) United States
(12) Patent Application Publication
(10) Pub. No.: US 2024/0307208 A1
(43) Pub. Date: Sep. 19, 2024

(54) Back support brace and method of wearing same

(71) Applicant: ADVANCED ORTHOPAEDICS, INC., Harleysville, PA (US)

(72) Inventor: Scott Geist, Hilton Head Island, SC (US)

(73) Assignee: ADVANCED ORTHOPAEDICS, INC., Harleysville, PA (US)

(21) Appl. No.: 18/445,359

(22) Filed: Jul. 31, 2023

Related U.S. Application Data
(60) Provisional application No. 63/576,898, filed on Mar. 13, 2023.

(51) Int. Cl. A61F 5/02

(52) U.S. Cl. CPC A61F 5/028

(57) ABSTRACT

The invention relates to a back support brace and a method of wearing the same. In a preferred embodiment, the back support brace includes a central brace portion formed to rest in support of a back of a person, a pair of adjustable brace portions configured to removably attach to the central brace portion on either side thereof, wherein each of the pair of adjustable brace portions include means to adjust the central brace portion in size according to size of the back of a person, and a removable pad or shell disposed upon the central brace portion to provide abutting support to the back of the person wearing the brace. The back support brace includes a pair of adjustable pull tabs secured to a length of cord extending from opposite sides of the central brace portion on each side for securing placement of the brace upon the user with each pull tab comprising a pull ring secured to a forward edge of an end sleeve formed having a pocket through which the cord is passed and held secured upon a pull tab adjusting member in the form of a notched panel sized to fit and be removable from the sleeve pocket so that the effective cord length may be adjusted by winding or unwinding the cord about the notched panel.

### CROSS-REFERENCE TO RELATED APPLICATION

This application claims the benefit of U.S. Provisional Application Ser. No. 63/576,898 filed Mar. 13, 2023 for a Back Support Brace and Method of Wearing Same.

### FIELD OF THE INVENTION

The present disclosure relates to a back support brace and a method of wearing the same. More specifically, the back support brace provides optimum compression around the waist of a user with adjustable pull tab means to improve fitted attachment of the brace and provide more comfortable support to the back.

### BACKGROUND OF THE INVENTION

Back pain is a very common problem faced by individuals during the course of their lives. Many back issues are treated with medicines or physiotherapy. In addition, back support braces may be worn by individuals to assist in healing of back injuries. Further, a back support brace promotes good posture, supports back and waist muscles when they are weak, alleviates pain and enables a patient to move around during recovery from any injury.

There are several patents heretofore issued on back support brace or belts that provide back panels or side panels along the belt and belt adjustment means to try and achieve an appropriate fit for the person wearing the same. However, it is extremely difficult to configure a brace with ideal fit without causing discomfort to the person wearing it. Moreover, most of the belts provide adjustment means that wear our very quickly or provide a very clumsy fit around the waist thereby causing discomfort.

For example, U.S. Pat. No. 11,324,622 discloses a prior art back brace that includes a back plate; left and right cord plates each slidably mounted to left and right sides of the back plate, respectively, and to which first and second flexible belt members are respectively secured, and a cord guide arrangement for each plate to particularly direct first and second cords. However, this prior art back brace is extremely cumbersome to operate due a structure having multiple plates, cords with a complex operational arrangement, and a rigid plate structure thereby making it extremely uncomfortable to wear for any extended duration of time. Further, such back brace may not be suitable for patients with certain orthopedic condition that require more flexibility in the back brace to adjust to the structure of the patients back.

In view of the above and other prior art patents, there is a need for a back support brace which can be worn for a long duration of time without discomfort, which is easy to operate and fits appropriately to any individual regardless of the waist size.

### SUMMARY OF THE INVENTION

The inventive concepts presented herein are illustrated in a number of different embodiments, each showing one or more concepts, though it should be understood that, in general, the concepts are not mutually exclusive and may be used in combination even when not so illustrated as such.

Accordingly, a general aspect of the present invention is to provide an improved back support brace for a person wearing same. The back support brace includes a central brace portion formed to rest in support of a back of a person and a pair of adjustable brace portions configured to removably attach to the central brace portion where each of the pair of adjustable brace portions include means to adjust size of the pair of adjustable brace portions according to size of the back of a person. The back support brace also includes a removable pad or shell resting on the central brace portion thereby providing support to the back of the person wearing the brace.

In one embodiment, the back support brace includes a pair of adjustable pull tabs extending from the central brace portion on either side and configured for providing optimal compression to the back when wearing the brace. Further, each of the pair of adjustable pull tabs include a pull tab size adjusting member configured and assembled to enable winding or unwinding of an associated cord upon the size adjusting member to facilitate placement of the pull tabs while obtaining the optimal compression before locking the pull tab on an exterior portion of the pair of adjustable brace portion.

In a related embodiment, the pair of adjustable pull tabs includes a pull ring secured to a forward edge of an end sleeve formed having a pocket through which the cord is passed and held secured upon the pull tab size adjusting member removable from the sleeve pocket to adjust the effective cord length by winding or unwinding the cord about the pull tab size adjusting member.

In another embodiment, the back support brace includes at least one side panel positioned on an anterior surface of each of the pair of adjustable brace portion wherein the side panel is configured to provide support to the back of the person.

In a further embodiment, the back support brace includes a central brace portion having means to accommodate an adjustable left brace portion and an adjustable right brace portion wherein the adjustable left brace portion and the adjustable right brace portion are configured for adjusting length of the portion depending on a waist size of a person. The back support brace also includes a removable back pad or shell configured for resting on the central brace portion to provide support to back of the person, and a pair of adjustable pull tab extending from the central brace portion configured for providing optimal compression to the waist when wearing the brace.

In yet another embodiment, the present invention provides a method of wearing a back support brace. The method includes the steps of adjusting size of a pair of adjustable brace portion according to the size of waist of a person, attaching the pair of adjustable brace portion to a central brace portion by inserting the adjustable brace portion inside the central portion, attaching a removable pad or shell on an anterior surface of the central brace portion and placing the removable pad on back of the person to provide support, placing the pair of adjustable brace portion around the waist for locking against each other, pulling a pair of pull tabs and wrapping a cord connected on each of the pull tabs around a notched panel to set the proper length of each of the pull tabs to provide optimal compression around the back, and locking the pull tabs on an exterior surface of each of the pair of adjustable brace portion for wearing the brace.

In an advantageous aspect, the back support brace of the invention is configured with multiple adjustable elements to ensure a proper fit for the person wearing the belt. Moreover, the pull tabs provide desired compression around the waist without a clumsy connection thereby ensuring comfortable and easy usage for the person.

To further clarify the advantages and features of the present invention, a more particular description of the invention will be rendered by reference to specific embodiments thereof, which is illustrated in the appended figures. It is appreciated that these figures depict only typical embodiments of the invention and are therefore not to be considered limiting of its scope.

### DETAILED DESCRIPTION OF THE INVENTION

Described herein are nonlimiting example embodiments of the present invention, which includes a back support brace and a method of wearing the same.

The various embodiments including the example embodiments will now be described more fully with reference to the accompanying drawings, in which the various embodiments of the invention are shown. The invention may, however, be embodied in different forms and should not be construed as limited to the embodiments set forth herein. Rather, these embodiments are provided so that this disclosure will be thorough and complete, and will fully convey the scope of the invention to those skilled in the art. In the drawings, the sizes of components may be exaggerated for clarity.

It will be understood that when an element or layer is referred to as being “on,” “connected to,” or “coupled to” another element or layer, it can be directly on, connected to, or coupled to the other element or layer or intervening elements or layers that may be present. As used herein, the term “and/or” includes any and all combinations of one or more of the associated listed items.

Spatially relative terms, such as “left brace portion,” “right brace portion,” or “central brace portion,” and the like, may be used herein for ease of description to describe one element or feature's relationship to another element(s) or feature(s) as illustrated in the figures. It will be understood that the spatially relative terms are intended to encompass different orientations of the structure in use or operation in addition to the orientation depicted in the figures.

The subject matter of various embodiments, as disclosed herein, is described with specificity to meet statutory requirements. However, the description itself is not intended to limit the scope of this patent. Rather, the inventors have contemplated that the claimed subject matter might also be embodied in other ways, to include different features or combinations of features similar to the ones described in this document, in conjunction with other technologies. Generally, the various embodiments including the example embodiments relate to a back support brace and a method of wearing the same.

Referring to the drawings, the following is a list of elemental parts and components of the present back support brace and those associated structural elements shown throughout the drawings and employed in connection with the present invention:
100 back support brace;
101 central brace portion;
101a removable pad or shell;
102 adjustable left brace portion;
103 adjustable right brace portion;
104 means to adjust length/size of brace portions;
105, 106 pair of side panels;
107, 108 pair of adjustable pull tabs;
109 cord for pull tabs;
110, 111 end sleeves;
110a,111a end sleeve pockets;
112, 113 notched insert panels;
112a,113a notch formations; and
114,115 ring members.

Referring now to FIG. 1, there is shown an exploded view of a back support brace (100) in accordance with an embodiment of the invention. The back support brace (100) includes a central brace portion (101), a removable pad or shell (101a), a pair of adjustable brace portion (102, 103) each having a means 104 to adjust length/size of the pair of adjustable brace portion (102, 103), a pair of side panels (105, 106) accommodated on the pair of adjustable brace portion (102, 103), and a pair of adjustable pull tabs (107, 108).

Referring to FIG. 1A in conjunction with FIG. 1, the central brace portion 101 of the assembled back support brace (100A) is formed to rest in support of a back of a person. The central brace portion (101) accommodates the removable back pad or shell (101a) to provide support to the back of the person. The removable pad or shell (101a) is contoured to ensure optimal fit for the person. The central brace portion (101) is configured to accommodate the pair of adjustable brace portion (102, 103) to form a belt-like wrap to be worn by the person.

In an exemplary embodiment of the present invention, the pair of adjustable brace portions (102, 103) includes an adjustable left brace portion (102) and an adjustable right brace portion (103) for adjusting the lengths depending on the waist size of the person wearing the belt. The pair of adjustable brace portion (102, 103) includes one or more sections (104) along the anterior surface of the brace portions that may be engaged on either side of the central brace portion (101) and fold the lengths of the left and right brace portions (102, 103) and thereby adjust the overall length of the belt-like wrap of the combined brace portions to overlap the waist of the person so that when the brace portions are engaged about the waist and then compressed about the front torso, firm back support may be applied by the central brace portion (101) and its pad or shell (101a) regardless of the size of the person.

Also in the present embodiment, the pair of adjustable brace portions (102, 103) are each attached to the central brace portion (101) through a set hook and loop fabric-like fasteners, such as those commercially available under the trademark VELCRO. The pair of side panels (105, 106) are positioned on the anterior surfaces of the adjustable brace portions (102, 103), with the side panel (105) being positioned on the anterior surface of the adjustable left brace portion (102) and the side panel (106) being positioned on anterior surface of the adjustable right brace portion (103). The pair of side panels (105, 106) are configured to provide support to the back of the person.

Referring to FIGS. 1B and 1C in conjunction with FIG. 1, the back support brace (100, 100B, 100C) is shown with the pair of pull tabs (107,108) made adjustable in accordance with the present invention. Each of the pair of adjustable pull tabs (107, 108) is connected to a length of a cord 109 and further includes an end sleeve (110, 111) with a pocket (110a, 111a) formed therein and sized to accommodate a notched insert panel (112, 113) that serves as a size-adjusting member for the back support brace (100). The cord 109 should be strong and durable in its construction and may have only some limited elasticity to help avoid fracturing or tearing under extended usage of the pull tabs (107,108). The notched insert panels (112, 113) are rectangular in configuration with notches (112a,113a) formed on opposite edges of the panel to engage the cord (109). The notches (112a, 113a) in the insert panels (112, 113) act to retain the cord (109) upon the insert panels and enable winding or unwinding of the cord around the panels and thereby adjust the lengths of the respective pull tabs (107,108) so that each may be moved into position of obtaining the optimal compression before locking the pull tabs on an external portion of the adjustable brace portions (102, 103). The pull tabs (107, 108) each further include a ring (114, 115) passing through and engaging the sleeve (110, 111) at the outer end of the pull tabs where the ring (114, 115) enables the person/user to hold and pull the pull tabs to a desired compression state before locking it. Accordingly, in a preferred embodiment, the size adjusting member (112, 113) is a notch insert panel having at least one notch (112a, 113a) configured to retain the cord (109) when the cord 109 is wound around the notch insert panel (112, 113). The lengths of cord 109 running along the left and right brace portions (102,103) are connected to the central brace portion (101) at their inner ends with the lengths of the cord routed forward to pass through and into the end sleeve (110, 111) and connect in engagement with the notched insert panels (112, 113) on each side of the back support brace (100).

Referring to FIGS. 2 and 2A, perspective view (200, 200A) of the left support brace (102) with the adjustment means is shown in accordance with an embodiment of the invention. The left support brace (102) includes sections on interior surface of the brace (102) that act as length adjustment means (104) for folding the brace to adjust length of the brace (102). Once the length is adjusted, the adjustable left brace (102) is inserted inside the cavity or slot of the central brace portion (101). Similarly, length of the adjustable right brace is adjusted depending on the waist of the person before inserting it inside a cavity or slot on other side of the central brace portion (101) thereby forming a belt to be worn by the person as shown in perspective view (200B) of FIG. 2B. The adjustable left brace (102) and the adjustable right brace (103) are connected to the central brace portion (101) through hook and loop fasteners.

Referring to FIG. 2C, a perspective view (200C) of a person pulling the pair of pull tabs (107, 108) to achieve the required compression is shown in accordance with an embodiment of the invention. Depending on the waist size of the person, the cords of the pull tabs (107, 108) are extended and wrapped around the size adjustment means/notched insert panel before inserting back inside the cover of the pull tabs to ensure a clean and easy fit. Once the desired compression is achieved, the pull tabs are connected to an exterior surface of the adjustable left (102) and right brace portion (103) through the hook and loop fastener as shown in perspective view (200D) of FIG. 2D.

While various elements of the back support brace including the adjustable left brace portion (102), the adjustable right brace portion (103), pull tabs (107, 108) are shown to be attached or connected through a hook and loop fastener, it shall be apparent to a person skilled in the art that other connections may be used for fixing these elements to the surface of the belt within the scope of this invention.

Variations of the type, form, size, and material of any of central brace portion, adjustable left brace portion, right brace portion, pull tabs may be of any of those described previously and used in any of the method described.

Obviously, other embodiments and modifications of the present invention will readily come to those of ordinary skill in the art having the benefit of the teachings presented in the foregoing description and drawings. Alternate embodiments of different shapes and sizes, as well as substitution of known materials or those materials which may be developed at a future time to perform the same function as the present described embodiment are therefore considered to be part of the present invention. Furthermore, certain modifications to the described embodiment that serve to benefit its usage are within the scope of the present invention. Accordingly, it is understood that this invention is not limited to the particular embodiment described, but rather is intended to cover modifications within the spirit and scope of the present invention including the appended claims.

### BRIEF DESCRIPTION OF THE DRAWINGS

The above contents of the present invention will become more readily apparent to those ordinarily skilled in the art after reviewing the following detailed description and accompanying drawings, in which:

FIG. 1 shows an exploded view of a back support brace in accordance with an embodiment of the invention;

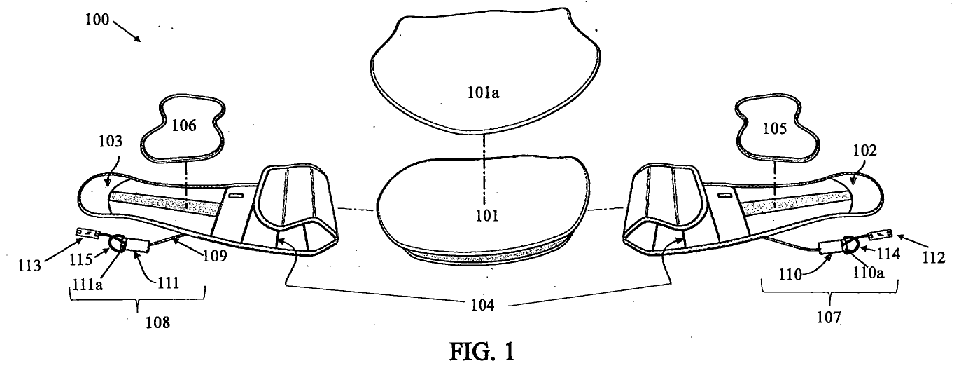

FIG. 1A shows a perspective view of the inner side of the back support brace assembled and laid out for use in accordance with an embodiment of the invention;

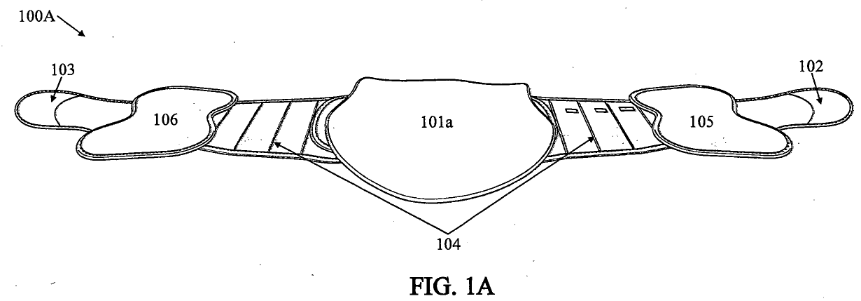

FIG. 1B shows a front view of the back support brace with a close-up view of one of the pair of the adjustable pull tabs for tightening and securing the brace around the waist in accordance with an embodiment of the invention;

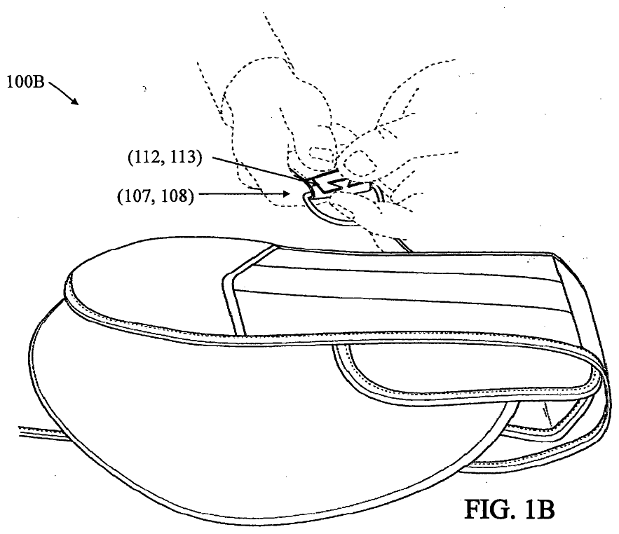

FIG. 1C shows the adjustable pull tab means with a notched insert panel used as a pull tab size adjusting member for adjusting the effective cord length in accordance with an embodiment of the invention;

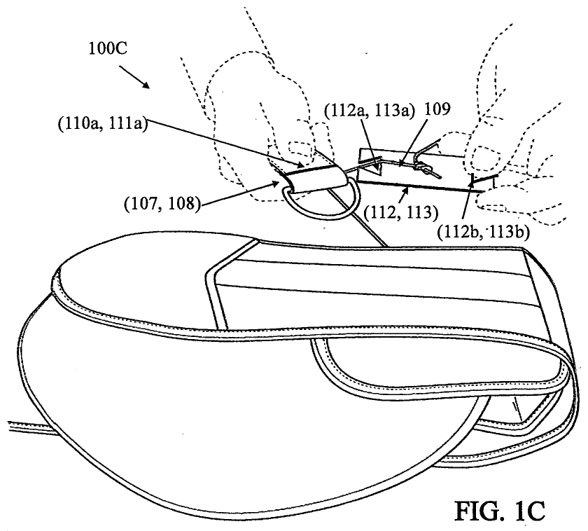

FIG. 1D shows the notched insert panel used as the pull tab size adjusting member with notches configured to retain the cord wrapped around the notch panel in accordance with an embodiment of the invention.

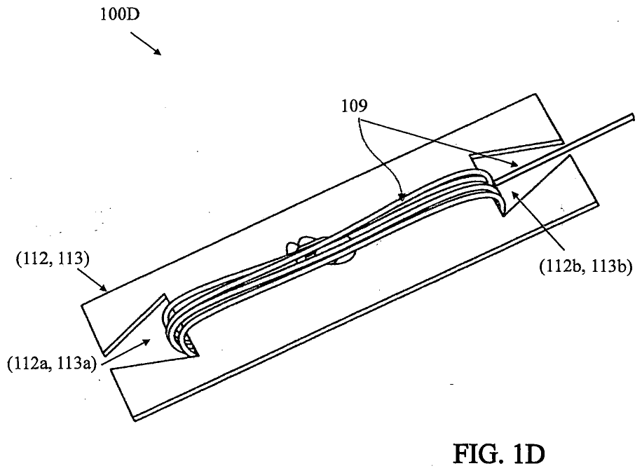

FIG. 2 shows a view of an adjustable left brace portion being folded around an anterior section of the adjustable left brace portion to adjust length of the portion before being accommodated inside a cavity or slot of a central brace portion of the back support brace in accordance with an embodiment of the invention;

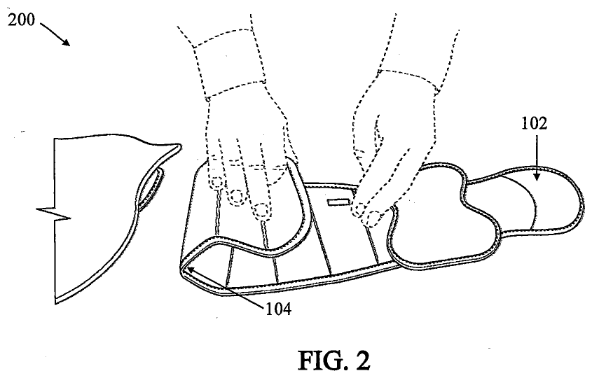

FIG. 2A shows a view of the adjustable left brace portion being inserted inside a cavity or slot of the central brace portion of the back support brace in accordance with an embodiment of the invention;

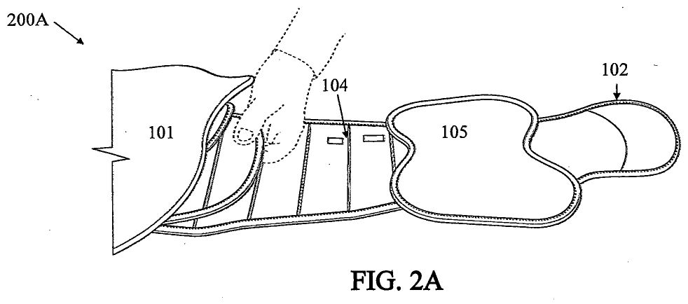

FIG. 2B shows a view of a back support brace worn by a person in accordance with an embodiment of the invention;

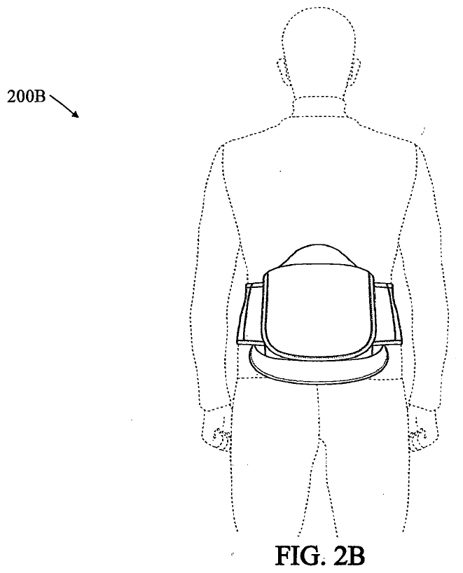

FIG. 2C shows a front view of the back support brace with pull tabs worn by the person in accordance with an embodiment of the invention; and

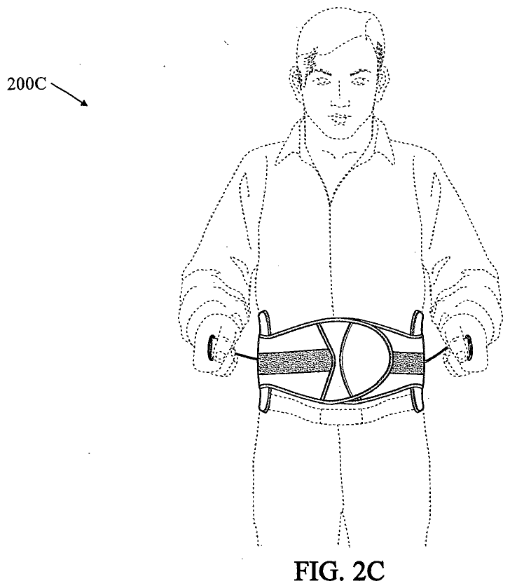

FIG. 2D shows a front view of the back support brace worn by the person in accordance with an embodiment of the invention.

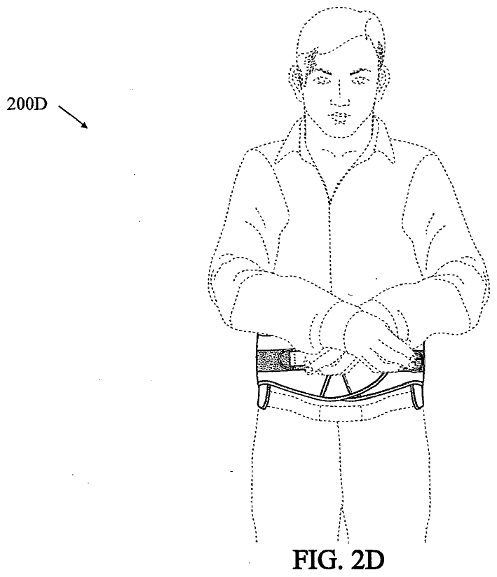

Further, skilled artisans will appreciate that elements in the figures are illustrated for simplicity and may not have been necessarily drawn to scale. Furthermore, in terms of the construction of the device, one or more components of the device may have been represented in the figures by conventional symbols, and the figures may show only those specific details that are pertinent to understanding the embodiments of the present invention so as not to obscure the figures with details that will be readily apparent to those of ordinary skill in the art having the benefit of the description herein.

What is claimed is:

1. A back support brace for a person wearing same, comprising:
a central brace portion formed to rest in support of a back of a person;
a pair of adjustable brace portions configured to removably attach to the central brace portion and extend on either side thereof for engagement about the waist of the person, the pair of adjustable brace portions including means for adjusting the extension thereof according to size of the person;
a removable pad resting on the central brace portion to provide abutting support to the back of the person wearing the brace; and
adjustable pull tab means operatively connected to the central brace portion and routed about the adjustable brace portions for providing fitted engagement of the brace portions about the waist and optimal compression to the back of the person when wearing the brace.

2. The back support brace of claim 1, wherein the adjustable pull tab means comprises:
a length of cord routed from the central brace portion and about the adjustable brace portions on either side thereof; and
a notched panel member connected to the length of cord and configured to enable winding or unwinding of the cord into a secured engagement upon the notched panel member.

3. The back support brace of claim 2, wherein the adjustable pull tab means further comprises:
an end sleeve formed having a pocket sized to hold the notched panel member therein with the cord passed therethrough and allow removal of the notched panel member from the sleeve pocket to adjust the cord length upon the notched panel member; and
a pull ring secured to a forward edge of the sleeve.

4. The back support brace of claim 3, wherein anterior surfaces along the adjustable brace portions are provided with hook and loop fasteners and the end sleeve of the pull tab means is provided with a hook and loop fastener surface on the outside thereof to allow releasably engagement with the adjustable brace portions along anterior surfaces thereof.

5. The back support brace of claim 1, wherein the pair of adjustable brace portions include one or more sections formed on an anterior surface thereof for folding the adjustable brace portions to adjust the extensions thereof according to the size of the back of the person.

6. The back support brace of claim 5, wherein the adjustable brace portions are attached to the central brace portion through a hook and loop fastener on their respective surfaces.

7. The back support brace of claim 1, further comprising:
at least one side panel positioned on an anterior surface of each of the pair of adjustable brace portions wherein the side panel is configured to provide support to the back of the person.

8. A back support brace for a person wearing same, comprising:
a central brace portion formed to rest in support of a back of a person;
a pair of adjustable brace portions formed to engage to the central brace portion on either side thereof and extend therefrom for engagement about the waist of the person, the pair of adjustable brace portions including means for adjusting the extension thereof according to size of the person; and
a pair of adjustable pull tabs operatively connected to the central brace portion and about the adjustable brace portions on either side for providing fitted engagement of the brace portions about the waist and optimal compression to the back of the person when wearing the brace.

9. The back support brace of claim 8, wherein each of the adjustable pull tabs comprise:
a length of cord routed from the central brace portion and about the adjustable brace portions on either side thereof;
a notched panel member connected to the length of cord and configured to enable winding or unwinding of the cord into a secured engagement upon the notched panel member;
an end sleeve formed having a pocket sized to hold the notched panel member therein with the cord passed therethrough and allow removal of the notched panel member from the sleeve pocket to adjust the cord length upon the notched panel member; and
a pull ring secured to a forward edge of the end sleeve.

10. The back support brace of claim 8, further comprising:
a removable pad disposed on the central brace portion to provide abutting support to the back of the person wearing the brace.

11. The back support brace of claim 9, wherein anterior surfaces along the adjustable brace portions are provided with hook and loop fasteners and the end sleeves of each of the adjustable pull tabs is provided with a hook and loop fastener surface on the outside thereof to allow releasable engagement with the adjustable brace portions along anterior surfaces thereof.

12. The back support brace of claim 10, wherein the pair of adjustable brace portions include one or more sections formed on an anterior surface thereof for folding the adjustable brace portions to adjust the extensions thereof according to the size of the back of the person.

13. The back support brace of claim 12, wherein the adjustable brace portions are attached to the central brace portion through a hook and loop fastener on their respective surfaces.

14. The back support brace of claim 13, further comprising:
at least one side panel positioned on an anterior surface of each of the pair of adjustable brace portions wherein the side panel is configured to provide support to the back of the person.

15. A method of wearing a back support brace upon the torso of a person, comprising:
positioning a central brace portion of the back support brace across the back area with a pair of adjustable brace portions extending on either side of the central brace portion;
adjusting the extended lengths of the brace portions according to the size of back area and waist of the person;
placing a pad or shell on the anterior surface of the central brace portion immediately abutting the back area to provide firm support;
wrapping the pair of adjustable brace portions together around the waist area in a closed engagement;
extending a pair of pull tabs each on a length of cord from the central brace portion and around the closed engagement of the brace portions and adjusting the cord length to comfortably tighten the brace portions about the waist; and
fastening the pull tabs along the exterior surfaces of the brace portions to maintain the brace portions in their tightened position around the waist thereby providing optimal compression around the back.
